(19) United States
(12) Patent Application Publication
(10) Pub. No.: US 2025/0166181 A1
(43) Pub. Date: May 22, 2025

(54) AUTOMATED EVALUATION OF RIGHT ATRIAL PRESSURE VIA MACHINE LEARNING

(71) Applicant: CALIFORNIA INSTITUTE OF TECHNOLOGY, Pasadena, CA (US)

(72) Inventors: Dominic YURK, GLENDALE, CA (US); Yaser S. ABU-MOSTAFA, PASADENA, CA (US); Arun PADMANABHAN, SAN FRANCISCO, CA (US); Geoffrey TISON, SAN FRANCISCO, CA (US)

(73) Assignee: CALIFORNIA INSITUTE OF TECHNOLOGY, Pasadena, CA (US)

(21) Appl. No.: 18/951,500

(22) Filed: Nov. 18, 2024

Related U.S. Application Data
(60) Provisional application No. 63/600,614, filed on Nov. 17, 2023.

(51) Int. Cl. G06T 7/00

(52) U.S. Cl. CPC G06T 7/0012; G06T 2207/10016; G06T 2207/10132; G06T 2207/20081; G06T 2207/30048

(57) ABSTRACT

Automated evaluation of RAP via machine learning is described herein. In one implementation, a method includes: obtaining first video scan data including multiple first video frames of an IVC of a subject, the multiple first video frames including a first video frame of the IVC while the subject is at rest, and a second video frame of the IVC while the subject is inhaling; determining, using a first trained model, based at least on the multiple first video frames, that the first video scan data corresponds to a sniff test of the IVC of the subject; and predicting, using a second trained model, based at least on the multiple first video frames, a RAP of the subject.

### CROSS-REFERENCE TO RELATED APPLICATIONS

The present application claims the benefit of U.S. Provisional Patent Application No. 63/600,614 filed on Nov. 17, 2023 and titled “AUTOMATED EVALUATION OF RIGHT ATRIAL PRESSURE VIA MACHINE LEARNING,” which is incorporated herein by reference in its entirety.

### BACKGROUND

A “sniff test” is a maneuver commonly performed by a patient while they are undergoing a transthoracic echocardiogram (TTE) and the results of which are currently evaluated manually by cardiologists. The health concern underlying the sniff test is heart failure (HF), a condition that results from a weakened heart muscle's inability to adequately pump blood to the body. Heart failure affects over 20 million people worldwide, and associated hospitalizations represent one of the greatest burdens to the healthcare system, driving up cost and resource utilization in wealthy and poor economies alike. A common symptom associated with HF is excessive fluid retention due to reduced cardiac output, referred to as volume overload (VO), which further worsens cardiac function and precipitates the spectrum of symptoms associated with HF (breathlessness, edema, chest pain, etc.). If left untreated, progressive VO can cause severe multi-system organ damage, and treatment may necessitate inpatient hospitalization. However, if detected early, VO can often be addressed with orally administered diuretics, thus obviating the need for hospitalization. Therefore, non-invasive methods that can safely and efficiently screen for volume overload are of great interest.

One of the most quantitative methods of assessing intravascular volume status is the measurement of intracardiac filling pressures via right heart catheterization (RHC), which is considered the “gold standard” for measuring intracardiac filling pressure in the right atrium, or right atrial pressure (RAP). In this procedure, a doctor inserts a flexible catheter with a pressure transducer at its tip into a vein in the neck, groin, or arm. The catheter is then gradually advanced through the venous system until it reaches the right side of the heart. This procedure yields highly accurate pressure measurements in the right atrium, right ventricle, and pulmonary artery. However, direct measurement of these pressures requires invasive cardiac catheterization, which is resource intensive, costly, and associated with a host of potential morbidities (infection, bleeding, arrhythmia, etc.).

A frequently used non-invasive surrogate is the estimation of RAP through ultrasound evaluation. Healthy patients have a low RAP of ˜3 mmHg. However, for patients with volume overload, there is an increase in the intravascular volume leading to an elevation of the RAP (˜15 mmHg or greater). Non-invasive RAP estimation is commonly performed based on the results of a sniff test conducted during ultrasound assessment of the inferior vena cava (IVC). In performing a sniff test, the ultrasound imaging probe is directed to generate an image of the IVC as it enters the right atrium from a sub-xiphoid window. The IVC diameter is first measured at rest, and then the patient is asked to sniff sharply in order to rapidly increase intrathoracic pressure. In a healthy patient with normal RAP, this increase leads to a brief collapse of the IVC. However, in a patient with an elevated RAP, the degree to which the IVC collapses is attenuated. To illustrate, FIG. 1A shows an ultrasound view of the IVC and right atrium at a resting state and during a sniff for a first patient. In this example, the IVC diameter was estimated to be 18 mm at rest and 2 mm during the sniff. The high degree of collapse during the sniff indicates that this first patient likely has a normal RAP. FIG. 1B shows an ultrasound view of the IVC and right atrium at a resting state and during a sniff for a second patient. In this example, the IVC diameter was estimated to be 24 mm at rest and 18 mm during the sniff. The low degree of collapse indicates that this patient likely has an elevated RAP.

Cardiologists have created standards to convert these two measurements, IVC diameter and collapsibility following a sniff, into an estimate of RAP that is frequently used, in concert with the physical examination and other clinical indicators, to make decisions regarding care. However, there are many potential sources of error in conducting sniff tests. For example, operators are advised to measure IVC diameter between 0.5 and 3 cm from the junction with the right atrium. Since the IVC tends to flare as it approaches this junction, the chosen point of measurement can impact the measured diameter significantly. Sniff tests parameters such as diameter can also mis-estimated if the imaging plane is not perfectly in line with the IVC, especially if the sniff causes the IVC to move out of the original imaging plane. In some cases, the IVC can even be confused with the abdominal aorta, which is located near the IVC and looks very similar in some patients but does not collapse significantly during a sniff regardless of RAP. In addition, multiple studies have found significant inter-operator variability amongst medical trainees, fellows, and emergency physicians when assessing IVC diameter and collapsibility, even after dozens of hours of training. The most reliable interpreters are generally considered to be experienced cardiologists, who have spent years evaluating many thousands of TTEs. However, while there is widespread access to ultrasound equipment capable of imaging the IVC for a sniff test, many medical centers do not have 24/7 access to cardiologists capable of making an assessment

As such, sniff test evaluation is subjective, and results can vary based on factors such as operator experience and image quality. Even in relatively well-controlled settings, prior studies have found that two doctors who independently evaluate a sniff test only produce concurring RAP estimates 70-80% of the time. Even if doctors agree with each other, they may not come to the correct answer. Other studies which compared the sniff test to gold-standard catheter measurements using RHC found that the sniff test only produced correct results about 50% of the time.

### SUMMARY

The technology described herein is directed to automated evaluation of RAP via machine learning.

In one embodiment, a non-transitory computer-readable medium has executable instructions stored thereon that, when executed by a processor, cause a system to perform operations comprising: obtaining first video scan data comprising multiple first video frames of an IVC of a subject, the multiple first video frames including a first video frame of the IVC while the subject is at rest, and a second video frame of the IVC while the subject is inhaling; determining, using a first trained model, based at least on the multiple first video frames, that the first video scan data corresponds to a sniff test of the IVC of the subject; and predicting, using a second trained model, based at least on the multiple first video frames, a RAP of the subject.

In some implementations, predicting the RAP of the subject is performed in response to determining that the first video scan data corresponds to a sniff test of the IVC of the subject.

In some implementations, the operations further comprise: prior to obtaining the first video scan data, obtaining second video scan data comprising multiple second video frames of the IVC; and determining, using a third trained model, based at least on the multiple second video frames, that an acceptable view of the IVC is being captured; and obtaining the first video scan data comprises obtaining the first video scan data in response to determining that the acceptable view of the IVC is being captured.

In some implementations, determining, using the third trained model, based at least on the multiple second video frames, that an acceptable view of the IVC is being captured, comprises: generating, using the third trained model, based at least on the multiple second video frames, a prediction comprising a confidence score that indicates a likelihood that the second video scan data is associated with an IVC class; and in response to determining that the confidence score meets a threshold, making a determination that an acceptable view of the IVC is being captured.

In some implementations, obtaining the first video scan data and the second video scan data comprises: capturing, using an ultrasound imaging device, a transthoracic echocardiogram of the subject.

In some implementations, the operations further comprise: obtaining multiple video scans, each of the multiple video scans corresponding to a medical imaging study; assigning multiple labels to the multiple video scans, each of the labels indicating whether or not a respective one of the video scans corresponds to a view of an IVC; and training, based on the multiple video scans and the multiple labels, a classification model as the third trained model.

In some implementations, determining, using the first trained model, based at least on the multiple first video frames, that the first video scan data corresponds to the sniff test comprises: generating, using the first trained model, based at least on the multiple first video frames, a prediction comprising a confidence score that indicates a likelihood that the first video scan data is associated with a sniff test class; and in response to determining that the confidence score meets a threshold, making a determination that the first video scan data corresponds to the sniff test of the IVC of the subject.

In some implementations, the operations further comprise: obtaining multiple video scans, each of the multiple video scans corresponding to a medical imaging study; assigning multiple labels to the multiple video scans, each of the labels indicating whether or not a respective one of the video scans corresponds to a view of an IVC during a sniff test; and training, based on the multiple video scans and the multiple labels, a classification model as the first trained model.

In some implementations, predicting, using the second trained model, based at least on the multiple first video frames, the RAP of the subject comprises: inputting the multiple first video frames into the second trained model; and generating, using the second trained model, a prediction output including the RAP.

In some implementations, the RAP of the prediction output is between 0 mmHg and 30 mmHg.

In some implementations, the RAP of the prediction output is 3 mmHg, 8 mmHg, or 15 mmHg.

In some implementations, the operations further comprise: obtaining multiple video scans, each of the multiple video scans corresponding to a sniff test of a subject; obtaining multiple RAP measurements, each of the RAP measurements corresponding to a respective one of the video scans; and constructing, based on the multiple video scans and the multiple RAP measurements, the second trained model.

In some implementations, the multiple video scans were captured by a plurality of different models of ultrasound imaging machines; and the multiple RAP measurements comprise a plurality of RAP estimates made by a plurality of different cardiologists.

In some implementations, each RAP measurement of the RAP measurements is an RAP estimate made by a physician based on the respective one of the video scans corresponding to the RAP measurement.

In some implementations, each RAP measurement of the RAP measurements is a RHC measurement made via RHC of a subject.

In some implementations, each of the RHC measurements corresponds to a respective one of the video scans made of a same subject within one month or less.

In some implementations, the multiple video scans comprise a first plurality of video scans and a second plurality of video scans; the multiple RAP measurements comprise a first plurality of RAP measurements and a second plurality of RAP measurements; each of the first plurality of RAP measurements is an RAP estimate made by a physician based on a respective one of the first plurality of video scans; and each of the second plurality of RAP measurements is a RHC measurement made via RHC of a subject, and corresponds to a respective one of the second plurality of video scans made of a same subject.

In some implementations, constructing, based on the multiple video scans and the multiple RAP measurements, the second trained model, comprises: pre-training, based on the first plurality of video scans and the first plurality of RAP measurements, an input model to estimate RAP based on an input video scan; and applying, based on the second plurality of video scans and the second plurality of RAP measurements, transfer learning to the input model to construct the second trained model.

In some implementations, applying transfer learning to the input model to construct the second trained model comprises: replacing an output layer of the input model with a new output layer to produce a new model; and training, using the second plurality of video scans and the second plurality of RAP measurements, the new model.

In some implementations, training the new model comprises assigning a lower learning rate to pre-existing layers of the input model present in the new model compared to the new output layer.

In one embodiment, a system comprises: a processor; and a non-transitory computer-readable medium having executable instructions stored thereon that, when executed by the processor, cause the system to perform operations comprising: obtaining first video scan data comprising multiple first video frames of an IVC of a subject, the multiple first video frames including a first video frame of the IVC while the subject is at rest, and a second video frame of the IVC while the subject is inhaling; determining, using a first trained model, based at least on the multiple first video frames, that the first video scan data corresponds to a sniff test of the IVC of the subject; and predicting, using a second trained model, based at least on the multiple first video frames, a RAP of the subject.

In some implementations, the system further comprises an ultrasonic imaging device configured to capture the first video scan data.

In some implementations, the ultrasonic imaging device is a portable ultrasonic imaging device.

In some implementations, the processor and non-transitory computer-readable medium are components of a mobile device; and the mobile device is communicatively coupled to the portable ultrasonic imaging device.

In some implementations, the system is a mobile device.

In some implementations, the operations further comprise: displaying, on a graphical user interface, the RAP that is predicted.

In one embodiment, a method comprises obtaining, at a computing device, first video scan data comprising multiple first video frames of an IVC a subject, the multiple first video frames including a first video frame of the IVC while the subject is at rest, and a second video frame of the IVC while the subject is inhaling; determining, at the computing device, using a first trained model, based at least on the multiple first video frames, that the first video scan data corresponds to a sniff test of the IVC of the subject; and predicting, at the computing device, using a second trained model, based at least on the multiple first video frames, a RAP of the subject.

In one embodiment, a non-transitory computer-readable medium has executable instructions stored thereon that, when executed by a processor, cause a system to perform operations comprising: obtaining multiple video scans corresponding to multiple sniff tests of multiple subjects; obtaining multiple RAP measurements, each of the RAP measurements corresponding to a respective one of the video scans; and constructing, based on the multiple video scans and the multiple RAP measurements, a trained model, the trained model configured to receive multiple video frames corresponding to a sniff test of a subject as an input, and generate, based on the input, a prediction output including a RAP of the subject.

In some implementations, at least one RAP measurement of the RAP measurements is a RHC measurement made via RHC of a subject.

In some implementations, the multiple video scans comprise a first plurality of video scans and a second plurality of video scans; the multiple RAP measurements comprise a first plurality of RAP measurements and a second plurality of RAP measurements; each of the first plurality of RAP measurements is an RAP estimate made by a physician based on a respective one of the first plurality of video scans; each of the second plurality of RAP measurements is a RHC measurement made via RHC of a subject, and corresponds to a respective one of the second plurality of video scans made of a same subject; and constructing, based on the multiple video scans and the multiple RAP measurements, the trained model, comprises: pre-training, based on the first plurality of video scans and the first plurality of RAP measurements, an input model to estimate RAP based on an input video scan; and applying, based on the second plurality of video scans and the second plurality of RAP measurements, transfer learning to the input model to construct the trained model.

In one embodiment, a non-transitory computer-readable medium has executable instructions stored thereon that, when executed by a processor, cause a system to perform operations comprising: obtaining first scan data associated with a first medical study of a heart of a subject; obtaining second scan data associated with a second medical study of the heart; converting, using one or more trained encoder models, the first scan data into a first tensor and the second scan data into a second tensor; grouping the first tensor and the second tensor into an unordered tensor; and transforming, using a trained transformer model, the unordered tensor into a final tensor encoding an overall state of the heart.

In some implementations, the first scan data comprises multiple first video frames associated with the first medical study of the heart; the second scan data comprises multiple second video frames associated with the second medical study of the heart; converting the first scan data into the first tensor comprises converting the multiple first video frames into the first tensor; and converting the second scan data into the second tensor comprises converting the multiple second video frames into the second tensor.

In some implementations, the first scan data and the second scan data correspond to a transthoracic echocardiogram of the subject.

In some implementations, the first scan data corresponds to an ultrasound scan of the heart, a CT scan of the heart, or an ECG of the heart; and the second scan data corresponds to another one of the ultrasound scan, the CT scan, or the ECG.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The present disclosure, in accordance with one or more implementations, is described in detail with reference to multiple figures. The figures are provided for purposes of illustration only and merely depict example implementations. Furthermore, it should be noted that for clarity and ease of illustration, the elements in the figures have not necessarily been drawn to scale. The figures are not exhaustive and do not limit the present disclosure to the precise form disclosed.

FIG. 1A shows an ultrasound view of the IVC and right atrium at a resting state and during a sniff for a first patient that likely has a normal RAP.

FIG. 1B shows an ultrasound view of the IVC and right atrium at a resting state and during a sniff for a second patient that likely has an elevated RAP.

FIG. 2 is a high level diagram depicting a system within which the technology described herein can be implemented to provide automated evaluation of sniff tests, including estimation of RAP, using machine learning, in accordance with some implementations of the disclosure.

FIG. 3 is a flow diagram illustrating an example method of providing an automated evaluation of a sniff test using machine learning, including estimating RAP, in accordance with some implementations of the disclosure.

FIG. 4 is an operational flow diagram depicting an example method of building a first model that predicts whether or not an IVC is present in a video scan, in accordance with some implementations of the disclosure.

FIG. 5 is an operational flow diagram depicting an example method of building a second model that predicts whether or not a sniff is present in a video scan, in accordance with some implementations of the disclosure.

FIG. 6 is an operational flow diagram depicting an example method that applies transfer learning techniques to build a third model that predicts an RAP from a sniff video scan, in accordance with some implementations of the disclosure.

FIG. 7 is a flow diagram illustrating a particular implementation of the method of FIG. 3 using three machine learning classification models, in accordance with some implementation of the disclosure.

FIG. 8 show four example ultrasounds scans that the IVC/sniff classification models were trained to distinguish, in accordance with particular implementations of the disclosure.

FIG. 9 includes plots showing the accuracy of cardiologist RAP estimates compared to true RAP values from right heart catheterization, with various time windows allowed between cardiologist and RHC measurements: unlimited time, 1 month, 1 week, or same day.

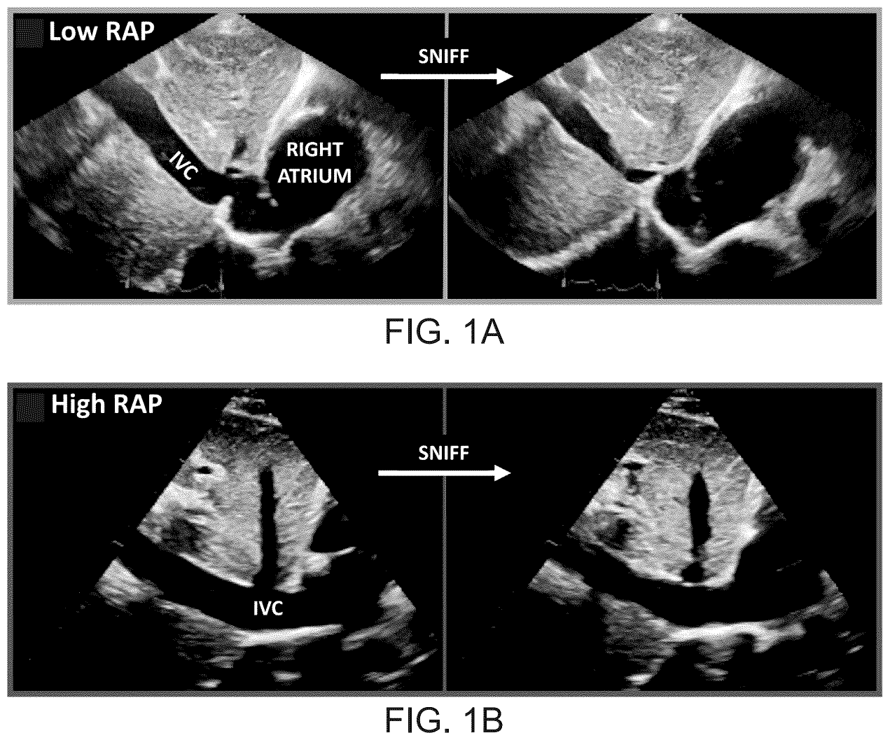

The figures are not exhaustive and do not limit the present disclosure to the precise form disclosed.

### DETAILED DESCRIPTION

There is a need for improved systems and methods for evaluating sniff test ultrasound imaging studies, including estimating RAP from the obtained images. In particular, there is a significant potential for machine learning-based techniques to provide a consistent interpretation of sniff tests without human operator variability and improve upon existing standards to attain better agreement with RHC measurements. To this end, implementations of the disclosure are directed to automated evaluation of sniff tests via machine learning.

In accordance with some implementations of the disclosure, multimodal techniques can be used to automatically evaluate a sniff test and estimate RAP. In such implementations, one or more trained models can be used to first identify a sniff scan as opposed to scans of other areas of the heart which are not immediately useful for this task. The one or more trained models can continuously run during imaging by a sonographer to determine whether a good view of the IVC and/or high-quality video of a sniff have been obtained. Thereafter, an additional trained model can be used to subsequently estimate patient RAP from the identified sniff scan. By virtue of employing the foregoing multimodal approach, the process of capturing a high quality sniff test can be simplified and semiautomated, potentially allowing for sniff tests to be performed anywhere with access to an ultrasound imaging device, without the requirement of a large medical center with a cardiology department.

In accordance with some implementations of the disclosure, the model trained to estimate RAP can be trained by leveraging a dataset including sniff scans of patients and corresponding RAP measurements of the same patients obtained using RHC. In some implementations, the model can first be pretrained on a large dataset of sniff scan evaluations by experienced cardiologists, and the model can subsequently be refined by applying transfer learning techniques based on a smaller dataset of right heart catheter measurements. By virtue of training the model in this manner, the trained model can match or even surpass cardiologist performance in estimating RAP from a sniff scan. These and other benefits from implementing the technology described herein are further described below.

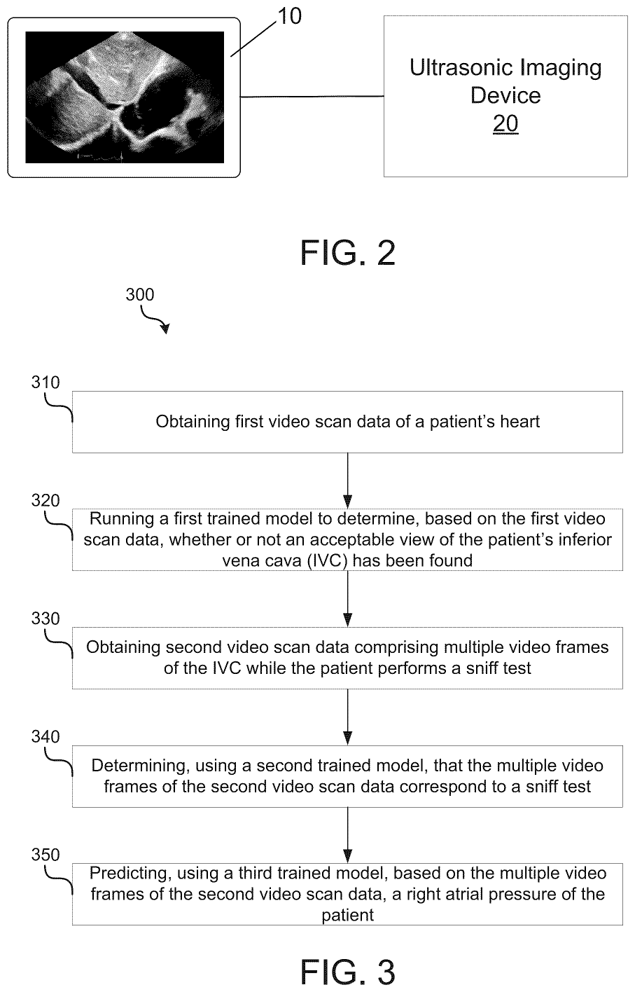

FIG. 2 is a high level diagram depicting a system within which the technology described herein can be implemented to provide automated evaluation of sniff tests, including estimation of RAP, using machine learning. As depicted, the system includes a sniff test evaluation device 10 in communication with an ultrasonic imaging device 20. During operation, the ultrasonic imaging device 20 is configured to scan a patient's/subject's heart as part of a medical imaging study (e.g., a TTE) of the patient/subject that includes a sniff test. Any suitable ultrasonic imaging device 20 can be used. For example, the ultrasonic imaging device 20 can be a portable ultrasound device such as the BUTTERFLY IQ or a desktop ultrasound imaging device. The video scan data, including the sniff test, captured by the ultrasonic imaging device 20 can be communicated from ultrasonic imaging device 20 to sniff test evaluation device 10 via a wired or a wireless communication link. For example, a wired USB link or a radio frequency link such as a Bluetooth® link or a Wi-Fi® link can be used to communicate the data.

The sniff test evaluation device 10 is configured to process the video scan data to provide an automated evaluation of the sniff test, including estimating the RAP of the subject. To this end, the sniff test evaluation device 10 can be configured to run one or more trained models during imaging by the sonographer to determine whether a good view of the IVC and/or high-quality video of a sniff test have been obtained. Once video of acceptable quality of a sniff test is obtained, the sniff evaluation device 10 can be configured to use an additional trained model to estimate RAP from the video scan data corresponding to the sniff test. Alternatively, in other implementations the sniff test evaluation device 10 can concurrently run all trained models. For example, the sniff test evaluation device 10 can concurrently determine whether or not a high quality video of the sniff test has been obtained while also estimating RAP from the current video stream. In yet other implementations, the ultrasonic imaging device can be configured to run the one or more initial trained models that determine whether good view of the IVC and/or high-quality video of a sniff have been obtained. In such implementations, the sniff test evaluation device 10 may only run the final trained model that estimates RAP from the sniff test scan.

In this particular example, sniff test evaluation device 10 is illustrated as a mobile device in communication with ultrasonic imaging device 20. The mobile device can be a smartphone, a tablet, a laptop, a smartwatch, a head mounted display (HMD), or other suitable mobile device configured to provide automated evaluation of the sniff test video scan data using the machine learning techniques described herein. In a particular implementation, the mobile device can run an application for performing automated evaluation of a sniff test using machine learning. The application can be configured to display data such as the video scan of the patient's heart, an indication of whether or not a good view of the IVC is being captured, an indication of whether high quality video of a sniff test is being obtained, and/or an estimate of RAP. In other implementations, a desktop computer or other device can be implemented as sniff test evaluation device 10.

In yet other implementations, sniff test evaluation device 10 and ultrasonic imaging device 20 can be integrated into the same device or system. For example, the functions and/or hardware of sniff test device 10 can be incorporated into ultrasonic imaging device 20. In some implementations, sniff test evaluation device 10 can be a device specifically dedicated to performing sniff test evaluation. For example, the device 10 can be designed with an integrated circuit that specifically performs the sniff test evaluation functions described herein. In such implementations, signal processing could take place in the analog domain. It should be appreciated from the foregoing description that the system used to performed automated evaluation of sniff tests using machine learning could be implemented in a variety of medical settings, including inpatient or outpatient settings, in large medical center or small medical office settings, and even potentially for at home use.

FIG. 3 is a flow diagram illustrating an example method 300 of providing an automated evaluation of a sniff test using machine learning, including estimating RAP, in accordance with some implementations of the disclosure. Method 300 can be performed, for example, as part of clinical screening to detect a potential heart condition. In some cases, method 300 can be performed as a follow up to a TTE study. In some implementations, some or all of the operations of method 300 can be performed in response to one or more processors executing instructions stored in one or more non-transitory computer-readable mediums. The one or more processors and the one or more non-transitory computer-readable mediums can be components of ultrasound imaging device 20 and/or sniff test evaluation device 10.

Operation 310 includes obtaining first video scan data of the patient's heart. The first video scan data can be obtained by scanning, using ultrasound imaging device 20, the patient's heart. A sonographer can use a commercially available ultrasound imager with embedded machine learning software to scan the patient's heart.

Operation 320 includes running a first trained model to determine, based on the first video scan data, whether or not an acceptable view of the patient's IVC has been found. For example, the first trained model can generate, based on multiple video frames corresponding to the first video scan data, a prediction comprising a confidence score that indicates a likelihood that the second video scan data is associated with an IVC class; and in response to determining that the confidence score meets or does not meet a threshold, make a determination that an acceptable or unacceptable view of the IVC is being captured. The first trained model can run continuously/recursively as the sonographer adjusts the view of the patient's heart. When an acceptable view of the IVC is being captured, the software can be configured to provide an indication/alert of the acceptable view on a display viewable by the sonographer.

Operation 330 includes obtaining second video scan data comprising multiple video frames of the IVC while the patient performs a sniff test. The second video scan data can be obtained from an ultrasound imaging device 20 as described above. Once a stable and/or high-quality view of the IVC is being obtained (e.g., as determined at operation 320), the patient can be prompted to inhale/sniff. The multiple video frames of the second video scan data can include one or more video frames of the IVC while the subject is at rest, and one or more video frames of the IVC while the subject is inhaling.

Operation 340 includes determining, using a second trained model, that the multiple video frames of the second video scan data correspond to a sniff test. The second trained model can be used to determine whether or not a high-quality video of the sniff was obtained; if not, the patient can prompted to sniff again, and operations 330-340 can repeat. In some implementations, the second trained model can generate, based at least on the multiple video frames, a prediction including a confidence score that indicates a likelihood that the first video scan data is associated with a sniff test class; and in response to determining that the confidence score meets or does not meet a threshold, making a determination that the first video scan data corresponds or does not correspond to a sniff test of the IVC of the patient.

In some implementations, operations 310-320 can be omitted. For example, the sonographer may not require guidance to identify the IVC and/or the software can be programmed not to perform the steps of determining that a good view of the IVC is being captured. Alternatively, the second trained model may have been trained to determine whether or not a sniff test of the IVC has been captured with a high enough accuracy that renders the use of the first trained model unnecessary.

Operation 350 includes predicting, using a third trained model, based on the multiple video frames of the second video scan, a RAP of the patient. For example, the software can be configured such that in response to determining that an acceptable/high-quality sniff scan has been obtained, the third trained model estimates the RAP from the sniff scan. A low RAP reading could indicate that the patient is not developing VO, while a medium to high RAP reading could indicate early VO and may merit referral to a specialist for further testing and treatment.

The multiple video frames can be input into the third trained model, and the third trained model can generate, based on the input, a prediction output including the estimated RAP. In some cases, the multiple video frames can be preprocessed into a format expected by the third model prior to being input into the third trained model. For example, color data can be removed, the video data can be unsampled or downsampled, pixel brightness can be adjusted, etc.

The estimated RAP of the prediction output can be in any format that is clinically useful. In one implementation, the third trained model can be configured to output a continuous RAP estimate. For example, the continuous RAP estimate can be anywhere from 0 to about 30 mmHg, which is the typical range of pressures that are reasonably possible for humans and closely matches the measurement output of some right heart catheters. In another implementation, the third trained model can be configured to output an RAP that is one of a set of discrete values indicative of an RAP level. For example, the third trained model can be configured to output an RAP that is either 3 mmHg, 8 mmHg, or 15 mmHg. An output in this format aligns with RAP measurements currently generated by cardiologists and endorsed by the American Cardiology Association and European Cardiology Association, where an output of 3 mmHg can indicate a normal RAP that does not necessitate further patient evaluation, an output of 8 mmHg can indicate an indeterminate RAP level where other metrics in concert with RAP can be used to determine whether further tests of the patient are appropriate, and an output of 15 mmHg can indicate an elevated RAP that necessitates further patient evaluation. In yet another implementation, the model can be configured to output human readable text indicating the state of the RAP. For example, the text output can indicate that the RAP is “low” or “normal”, that the RAP is “high” or “elevated”, or that the RAP is “medium” or “indeterminate”.

### Training

Large and preexisting datasets from one or more medical centers, including video data of sniff tests of patients and RAP measurements of those patients, can be used to train the models described herein. The data can include echocardiogram video data, associated measurements, and metadata. In some implementations, the video data can be a subset of relevant video data obtained from TTE studies, including the video data relating to patient IVCs and/or sniff tests. To improve the robustness/predictive capabilities of the trained models, the datasets can incorporate a diversity of data, including video scans captured using a plurality of different models of ultrasound imaging devices, RAP estimates from sniff scans made by a plurality of different cardiologists or other medical professionals qualified to estimate RAP, RAP measurements made using a plurality of different RHC devices, and data corresponding to patients spanning a wide range of demographics and medical conditions.

In some implementations, a first model that predicts whether an acceptable IVC view is present can be trained by obtaining multiple video scans, each of the multiple video scans corresponding to a medical imaging study; assigning multiple labels to the multiple video scans, each of the labels indicating whether or not a respective one of the video scans corresponds to a view of an IVC; and training, based on the multiple video scans and the multiple labels, a classification model. Given an input video, the trained first model can output a prediction indicating whether or not an IVC is present in the video.

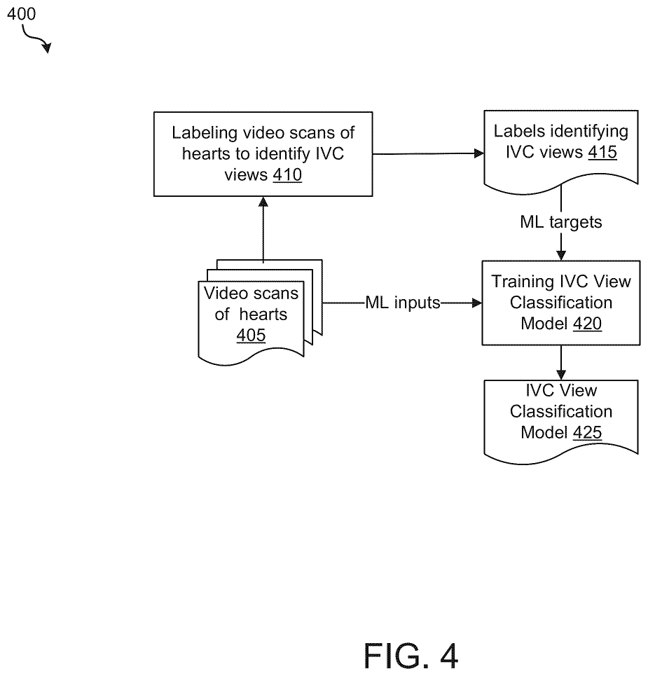

For example, FIG. 4 is an operational flow diagram depicting an example method 400 of building a first model (i.e., IVC view classification model 425) that predicts whether or not an IVC is present in a video scan. Operation 410 includes labeling video scans of hearts 405 to obtain labels 415 identifying IVC views. In some implementations, training data for the first model that identifies whether or not an IVC is present in a video scan can be generated by manually, semi-automatically, and/or automatically labeling video scans of hearts for whether or not an IVC is present in the scan. For example, during manual labeling/tagging, a user may utilize one or more controls or tools for adding labels. Each video scan (e.g., one or more video frames) can be tagged as being or not being an acceptable IVC view. In some cases, a binary tag can be used.

Operation 420 includes training the IVC view classification model 425 based on a training dataset including an input dataset and a target dataset. The input training dataset can include at least some of the video scans 405. The target training dataset may include at least some of the labels 415. In this case, the model 425 can be trained to extract features from an input video scan (e.g., one or more video frames), and output a target prediction of whether or not the video scan corresponds to an IVC view. The IVC view classification model 425 can also output a confidence score associated with each prediction. In particular implementations, a binary classification model can be used to identify IVCs. Training can be performed using the X3D-M architecture or other suitable architecture for ultrasound image classification.

In some implementations, a second model that predicts whether or not a sniff scan is present in a video scan can be trained by obtaining multiple video scans, each of the multiple video scans corresponding to a medical imaging study; assigning multiple labels to the multiple video scans, each of the labels indicating whether or not a respective one of the video scans corresponds to a view of an IVC during a sniff test; and training, based on the multiple video scans and the multiple labels, a classification model. Given an input video, the trained second model can output a prediction indicating whether or not a sniff scan is present in the video.

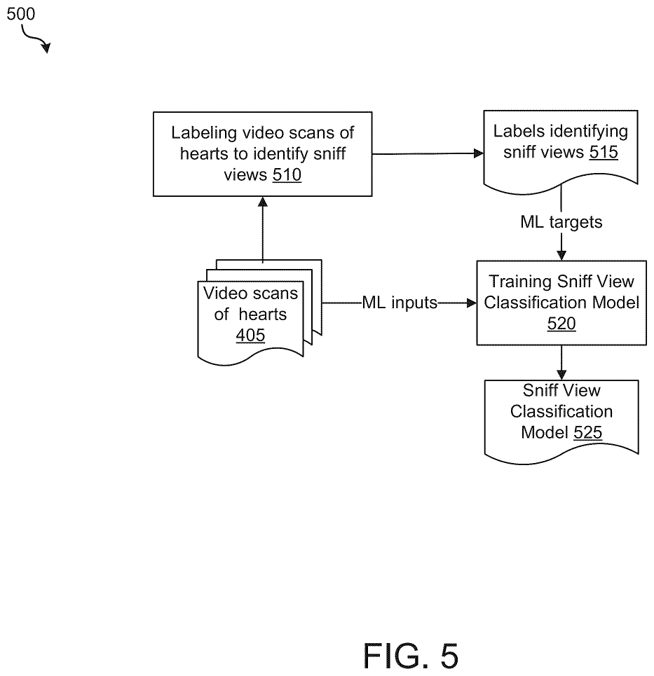

For example, FIG. 5 is an operational flow diagram depicting an example method 500 of building a second model (i.e., sniff view classification model 525) that predicts whether or not a sniff is present in a video scan. Operation 510 includes labeling video scans of hearts 405 to obtain labels 515 identifying sniff views. In some implementations, training data for a second model that identifies whether or not a sniff is present in a video scan can be generated by manually, semi-automatically, and/or automatically labeling video scans of hearts for whether or not a sniff scan is present in the scan. As depicted by method 500, the video scan data used to train the first model can be the same video scan data used to the train the second model. In other implementations, other video scan data can be used to train the second model.

Operation 520 includes training the sniff view classification model 525 based on a training dataset including an input dataset and a target dataset. The input training dataset can include at least some of the video scans 405. The target training dataset may include at least some of the labels 515. In this case, the model 525 can be trained to extract features from an input video scan (e.g., one or more video frames), and output a target prediction of whether or not the video scan corresponds to a sniff. The sniff view classification model 525 can also output a confidence score associated with each prediction. In particular implementations, a binary classification model can be used to identify whether a sniff test was present. Training can be performed using the X3D-M architecture or other suitable architecture for ultrasound image classification.

In some implementations, training data for a third model that estimates RAP from a sniff test video scan can include multiple video scans that have been previously identified as corresponding to sniff tests of multiple subjects, and multiple RAP measurements corresponding to the multiple video scans. The multiple video scans can be obtained based on video scans that have been previously labeled as corresponding to a sniff test as described above. The third model can be trained by obtaining multiple video scans, each of the multiple video scans corresponding to a sniff test of a subject; obtaining multiple RAP measurements, each of the RAP measurements corresponding to a respective one of the video scans; and constructing, based on the multiple video scans and the multiple RAP measurements, the model.

In some implementations, the multiple RAP measurements corresponding to the multiple video scans can include RAP measurements made by physicians/cardiologists based on the video scans of the dataset. In some implementations, by training the model on a diversity of data (e.g., data corresponding to a diversity of ultrasound imaging devices, cardiologist RAP estimates, patients, etc.), model performance can be improved. In particular implementations, further described below, it was observed by the inventors that a model trained on sniff scan evaluations generated by experienced cardiologists was able to generate statistically equivalent evaluations to the cardiologists on an out-of-sample test set.

In some implementations, the multiple RAP measurements corresponding to the multiple video scans can additionally or only include RHC measurements made via RHC of the subjects of the video scans. RHC is generally not performed at the same time as the sniff test video scans of patients, and there is some chance that the patient's true RAP can change significantly during the intervening time. However, as further described below, it was observed by the inventors that RHC measurements of RAP made of patients within a certain time window of capturing the video scans did not significantly impact the predictive accuracy of the trained model. In particular implementations, the time window can be about 1 month or less, e.g., each of the RHC measurements corresponds to a respective one of the video scans made of a same subject within about one month or less. In other implementations, the time window used to determine the cutoff for the training dataset can be longer or shorter.

The third model can be trained using any suitable video-processing machine learning architecture. In particular implementations, the SlowFast model architecture can be used, which works by dividing the flow of video data into “slow” and “fast” lanes. The slow lane is only shown a fraction of frames in the full video and has a high number of convolutional channels. The high channel count gives the model a lot of power to identify relatively static features, while the downsampling in frame count keeps computational load at a tractable level. The fast lane, in contrast, is shown the full set of video frames but has a relatively low number of convolutional channels. Seeing all frames allows this section of the model to detect quicker motions in the video, while the low channel count controls computational load. This architecture can be particularly suited/advantageous for RAP estimation as described above, which requires identifying both slow features (IVC location and resting diameter) and fast features (sniff-induced collapse) to make an accurate classification.

In some implementations, transfer learning techniques can be applied to refine a third model, initially trained using a large dataset of sniff scan evaluations generated by experienced cardiologists/medical professionals, with a smaller dataset of right heart catheter measurements.

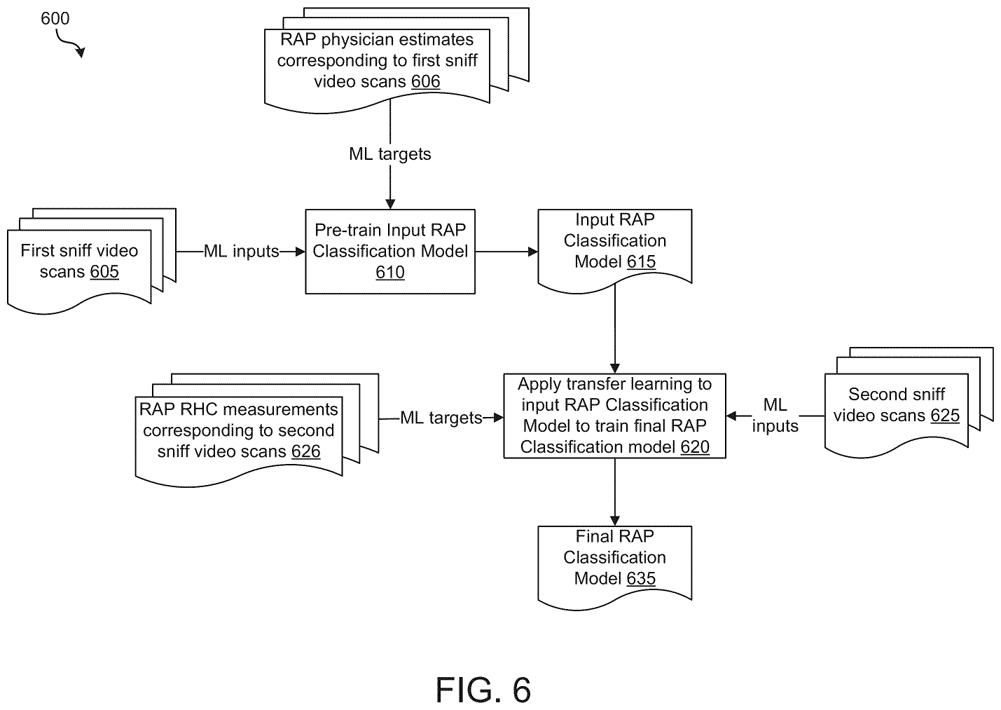

To illustrate, FIG. 6 is an operational flow diagram depicting an example method 600 that applies transfer learning techniques to build a third model that predicts an RAP from a sniff video scan, in accordance with some implementations of the disclosure. Operation 610 includes pre-training, based on a first set of sniff video scans 605 and a first set of RAP measurements 606 associated with physicians estimates of RAP from the first set of video scans, an input RAP classification model 615 model that estimates RAP based on an input video scan. Operation 620 includes applying, based on a second set of sniff video scans 625 and a second set of RAP measurements 626 obtained from RHC, transfer learning to the input model 615 to construct the final RAP classification model 635. Such transfer learning techniques can be particularly advantageous in cases where there is an insufficient amount of data available to train the third model using only RHC measurement data corresponding to video scans of sniff tests.

In particular implementations, further described below, it was observed by the inventors that an RAP estimation model fine-tuned using RHC RAP estimates was able to surpass cardiologist performance in some respects; in particular, it had a substantially lower rate of false negatives, i.e., yielding a low-RAP estimate for a patient with high RAP. It should be noted that a false negative is the most problematic type of error for a screening tool, as it could result in a sick patient being sent home with a clean bill of health.

In some implementations, the training data of the aforementioned models (e.g., IVC view classification model 425, sniff view classification model 525, and final RAP classification model 635) can be augmented. For example, the video scans can be augmented with random rotations, brightness scaling, contrast scaling, saturation scaling, and the like.

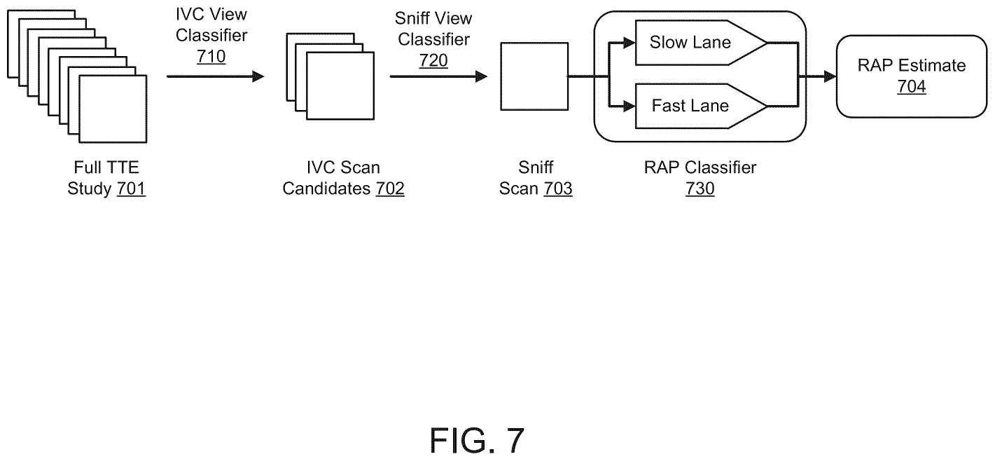

FIG. 7 is a flow diagram illustrating a particular implementation of method 300 using three machine learning classification models, in accordance with some implementation of the disclosure. Beginning with a full TTE study 701, an IVC view classifier 710 (e.g., IVC view classification model 425) identifies scans 702 in the study that show the IVC. Next, a sniff view classifier 720 (e.g., sniff view classification model 525) identifies sniff scan 703 as which of the IVC scans 702 is most likely to show a sniff test. Thereafter, an RAP classifier 730 (e.g., RAP classification model 635) analyzes the sniff scan 703 to determine an RAP estimate 704. In this particular implementation, the RAP classifier 730 uses the SlowFast architecture to determine an RAP estimate.

### Experimental and Simulation Results

Particular applications of the techniques described herein to construct three machine learning classification models to estimate RAP, and their associated results during application of the models to echo imaging studies to estimate RAP are described below. It should be appreciated that the forthcoming discussion describes some particular example implementations and that one having skill in the art would understand alternative implementations of the technology in view of the disclosure.

A database of echocardiograms performed in a cardiology division over several years, along with associated measurements, was utilized to construct datasets for machine learning. The high-level unit of data used in these particular implementations was the TTE study, which can include up to 200 individual scans which are taken in sequence on the same patient to gain many views of the heart and its surrounding vessels from different angles. Interpretations of these studies, such as RAP estimates, were made for the study as a whole. The data was split among three separate datasets with varying degrees of mutual overlap:
Dataset 1: Raw video data and associated metadata from echocardiogram studies. This covered 182077 studies on 48481 different patients, for a total of over 11 million individual scans. Video data from the scans was pre-processed to remove patient information, doctor's notes, Doppler velocity traces, etc. so only actual ultrasound video remained.
Dataset 2: RAP estimates made by cardiologists based on the sniff test. This covered 51324 studies on 35003 different patients.
Dataset 3: Gold-standard RAP measurements from right heart catheterization. This contained 9001 individual readings from 5585 different patients. These catheter measurements were independent of ultrasound measurements, and each patient may or may not have received a TTE.

TTE studies were removed from first and second datasets in their entirety if they met any of the following criteria:
Multiple different sniff-based RAP estimates were recorded for the same study.
The recorded study type noted a stress test, pediatric or fetal subject, trans-esophageal or intracardiac scanning, or patient on a ventilator. None of these study types are representative of how a standard TTE sniff test would be evaluated.
99.7% of remaining studies were conducted on one of 4 models of ultrasound machine; the remaining 0.3% of studies were eliminated.

Furthermore, individual scans were removed from the first dataset if they met any of the following criteria:
Video pre-processing failed.
The scan was less than 20 frames long. Such video clips are too short to contain a complete sniff test.
Physical pixel size was not recorded in metadata. This made it impossible to measure IVC diameter in real units.
Physical pixel size was in the lower or upper 5th percentile of pixel scale. This narrowed the total range of pixel scales from (0.002, 5.2) centimeters to (0.074, 0.168) centimeters. Extreme pixel sizes imply extreme ultrasound settings for parameters like scanning frequency and depth, which would generally not be used for viewing the IVC.
Color Doppler mode was enabled. Sniff scans are rarely taken with Color Doppler enabled, and extra splashes of color in a small subset of data would be likely to confuse ML models.

RHC measurements were excluded from the third dataset if they were less than 0 or greater than 30 mmHg, as these represent non-physical values that would have resulted from bad setup or calibration of the catheter.

The RAP estimates from the second dataset were not always made in the same format. While the large majority of RAP estimates in 2016 and later were recorded in the 3/8/15 format, prior to this, cardiologists in the dataset primarily used a different standard to estimate RAP as either 5, 10, 15, or 20 mmHg. Because these two standards were based on different sets of measurements and thresholds, they were not interconvertible. The inventors focused on the first category (3/8/15 format), as it is both the largest and aligned with modern clinical practice. A study is useful for machine learning training if it is associated with both input data (raw ultrasound video) and target data (an RAP value). After applying the data exclusion criteria described above, 16823 studies remained, representing the full breadth of our possible training data. Out of the 16823 remaining studies, 78.5% had an estimated RAP of 3 mmHg, 13.8% had 8 mmHg, and 7.8% had 15 mmHg.

Right heart catheterization is an entirely separate procedure from TTE. Patients who receive a TTE study may never receive an RHC (and vice versa), and patients that do receive both measurements will generally not have them taken simultaneously or even on the same day. As such, the inventors developed techniques to align the third dataset to the rest of the data. Out of the 5585 patients represented in the third dataset, 2299 of them had at least one TTE study in the first dataset, which may have been from any date. TTE studies and RHC measurements were paired in a 1:1 mapping such that only the closest-in-time RHC measurement for each TTE study was kept, leaving a total of 3483 RHC/TTE data pairs. After applying the data exclusion criteria described above, 2586 of these studies remained.

The next factor to consider was time separation between the echo and RHC measurements. Since the two measurements were not taken at the same time, there was some chance that the patient's true RAP changed significantly during the intervening time. If the two measurements were taken on the same day, this was relatively unlikely; if they were taken years apart it was significantly more likely. To narrow the data, a maximum allowable interval between the RHC and echo measurements was determined in order to be considered a valid data point. Based on analysis of cardiologist accuracy at different time intervals the inventors discovered an optimal cutoff of about 1 month (30 days), which left a total of 1739 studies which could be possibly used for RHC training data. Of these, 527 were labeled with an RAP estimate by cardiologists. The remaining 1212 were not, which could indicate that either a) a sniff test was not performed, or b) a sniff test was performed, but for unknown reasons a corresponding RAP estimate was not recorded.

A final consideration was how to determine the “accuracy” of cardiologist or machine learning RAP estimations (which are categorical) when comparing to RHC measurements (which are continuous). The inventors elected to put edge case readings into the lower of the two possible bins. Thus, an RHC value in the range [0, 5] was binned as “3,” a value in the range (5, 10] was binned as “8,” and a value in the range (10, 30] was binned as “15.” Continuous outputs from regression machine learning models were binned in the same way, allowing for direct accuracy comparisons between any combination of outputs from RHCs, cardiologist estimations, categorical ML models, and regression ML models.

Prior studies have found that physicians using the sniff test can often make errors in predicting RAP, as compared to gold-standard RHC measurements. These errors can stem from one of three sources: (1) The interpreter made an error in evaluating the sniff test; (2) the sniff scan contained sufficient information to accurately estimate RAP, but the measurements and thresholds were not the correct way to perform this estimate; or (3) the sniff scan did not contain sufficient information to accurately estimate RAP, no matter what analysis was performed. While training on a large body of sniff scans paired with cardiologist RAP estimates may partly mitigate type (1) error by averaging operator variability over many interpreters; however, no amount of such data could address type (2) or (3) errors.

By virtue of making use of RHC data, type (1) and (2) errors can be eliminated by removing human interpretation variability as well as potential issues with following conventional physician sniff test standards for estimating RAP from an ultrasound scan of the IVC. As further described below, by virtue of combining training on cardiologist RAP estimates as well as ground-truth RHC values, cardiologist performance at predicting true RAP was in some instances surpassed.

While a full TTE study can contain up to 200 individual scans, in general only about 2-4 scans show the IVC and only about 1-2 of these contain a sniff. Although the ultrasound technician knew which scans contained a sniff at the time of acquisition, these labels (or any other labels related to what was being viewed in each scan) were not recorded in first dataset. To address, the inventors developed an initial screening process to isolate the sniff scans in each study. The 16823 studies mentioned above contained a total of over 800,000 scans that passed the exclusion criteria and could potentially be sniffs. As this was too many scans to screen manually, a front-end machine learning model was trained to screen for sniff scans.

An IVC/sniff identification model was developed by generating a manually labeled set of scans for training. 1243 scans that had been marked as “subcostal” for a different study were used. This label included both IVC scans and other non-IVC views of the surrounding area. This first set of scans was reviewed to identify 420 that contained an IVC. Using this dataset, an initial binary classification ML model was trained to identify IVCs using the X3D-M architecture with default hyperparameters, which had previously been found to perform well for other ultrasound image classification tasks. To further build the training set, this initial model was used to classify many additional random scans out of the remaining 800,000, and 2757 were selected which were identified by the model (either correctly or incorrectly) as IVCs to add to the dataset.

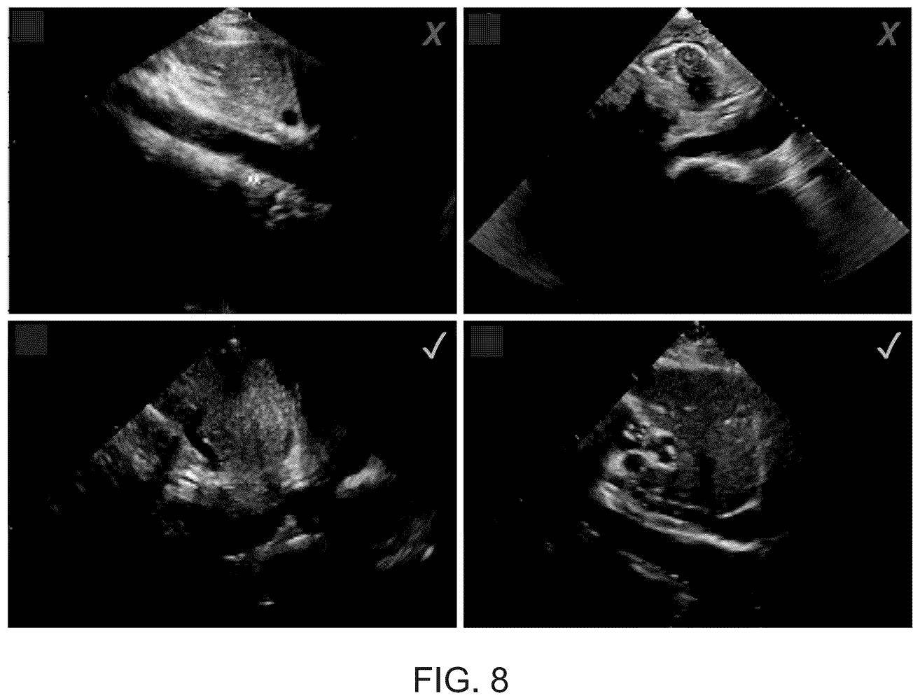

This set of 4000 scans was manually reviewed and labeled each as either 1) not a view of an IVC (2453 scans), 2) a view of an IVC without an obvious sniff (708 scans), or 3) a view of an IVC with a sniff (839 scans). Because most of the training videos were scans that had been classified as an IVC by the initial model, many of the type (1) scans were views that shared similarities to IVCs that caused them to be misclassified. The goal of this was to present the final IVC/sniff classification models with many challenging training cases, enhancing their ability to distinguish true IVCs from lookalikes. Four example, FIG. 8 show four example ultrasounds scans that the IVC/sniff classification models were trained to distinguish. The illustrated scans could be confused with an IVC, or are in fact IVCs but are hard to identify. The top left scan shows a vessel sloping in the correct direction with a darker space to the right, but it is the abdominal aorta rather than the IVC. The top right scan is from another region of the heart, but the dark area in the middle right happens to resemble an IVC starting to expand into the right atrium. The bottom left scan is an IVC, but the scan is quite noisy and a lot of the vessel and atrium are obscured. The bottom right scan an IVC with an artery next to it. This artery would not collapse during a sniff, which could confuse the sniff classifier.

To use this larger labeled dataset, the 4000 scans were divided into 2800 for training and 600 each for validation and testing. A binary classification X3D-M architecture was trained to distinguish type (3) scans from types (1) and (2). Training scans were augmented by random rotation and random scaling of brightness, saturation, and contrast on each epoch. Multiple training runs were conducted while varying the hyperparameters of optimizer type, learning rate, number of sampled frames, degree of augmentation, and dropout rate. The final model was chosen by maximizing validation accuracy. Table 1 shows the out-of-sample performance of the sniff/no sniff binary classification model. Grayscale cells represent the raw count of correct/incorrect predictions.

As illustrated, despite the relatively small training set and deliberate selection of challenging training and test cases, the sniff classification model still performed well when presented with out-of-sample scans. However, analysis of the results showed that the model was more likely to classify true sniffs as non-sniff scans when the degree of collapse induced by the sniff was small. This made intuitive sense, as a full collapse of the IVC could be seen very easily while a slight collapse may have been easier to miss and could potentially have been confused with a simple shift in probe positioning or fidgeting of the subject. This created the potential problem that sniffs with low collapse were more likely to come from patients with high RAP, so the model would be biased against identifying sniffs in such patients even though RAP was not part of the training data. As patients with an RAP estimate of 15 mmHg made up only 7.8% of the total training set, it was preferable not to disproportionately exclude them.

The inventors discovered that this problem can be addressed by training a separate binary classification X3D-M model to identify IVC views without regard to whether or not a sniff was present (i.e., distinguishing type (1) from types (2) and (3)). Table 2 shows the out-of-sample performance of the IVC/no IVC binary classification model, where gray cells represent the raw count of correct (light gray)/incorrect (dark gray) predictions.

In this particular implementation, the results of these classifier models were combined to identify sniffs in a 2-step procedure: 1) run the IVC classifier on all scans in a study. Identify a scan as an IVC “candidate” if the classifier's probability output is at least 20%. If no IVC candidates are found, reject the study; and 2) run the sniff classifier on all IVC candidates from the study. The candidate with the highest probability output from the sniff classifier is identified as the representative sniff scan for the study, even if that sniff probability is quite low.

Out of the 16823 studies remaining from the initial data selection procedure, 993 were excluded as having no IVC candidates. The remaining 15830 studies were assigned a representative sniff scan and represented the full dataset used for model training and evaluation. Of these studies, 79.1% had an estimated RAP of 3 mmHg, 13.4% had 8 mmHg, and 7.5% had 15 mmHg. This is very similar to the original split of 78.5%/13.8%/7.8%, indicating that the inventors did not introduce a substantial bias towards any particular class of RAP in our sniff identification procedure.

It is worth noting the diversity of the dataset. The 15830 studies included data from 11869 patients using one of four different models of ultrasound machine. Patients ranged from 18 to 102 years old and spanned a wide range of medical conditions. Study evaluations were conducted by a total of 45 different physicians, and 20 of these physicians evaluated over 100 studies. This diversity of data is a strength of this study; rather than training to match the judgement of a single physician reading off of a single ultrasound machine under controlled conditions, the training incorporated the full complexity of real-world medical data, making it far more likely to generalize to future real-world applications.

Inspection of a random subset the 15830 selected sniff scans revealed that a substantial majority did appear to be clear IVC scans, but roughly 25% were either low-quality IVC scans (where a significant portion of the vessel was noisy or obscured) or mis-selected scans that were not views of the IVC at all. To get a sense for the impact of these scans on model performance, the inventors also compiled a “high-quality” dataset via the same procedure described above, with the difference that the cutoff for IVC classification probability was 90%. This cutoff eliminated roughly 65% of studies in the original dataset; out of what was left, inspection of a subset found that 98% of scans seemed to be high-quality IVC images. While this rate of data discarding was too high to be useful for training, it was useful for evaluating model test performance on a closer-to-ideal dataset.

The same sniff selection described above was also applied to the 1739 TTE studies with an associated RHC measurement within 30 days of the study date. This yielded 932 studies that were identified as containing an IVC. This relatively low IVC identification rate may be due to the fact that 1212 out of the original 1739 studies did not have a recorded echo-based RAP estimate, indicating that many of these studies likely did not contain a sniff. The 932 identified sniff scans were manually screened to remove any non-IVC views (IVC views without an obvious sniff were left in), leaving 866 studies remaining. This 93% success rate indicates that the sniff identification process performed fairly robustly. For these studies, 35% had a catheter pressure in the range [0,5], 29% were in (5,10], and 36% were in (10, 30].

Of the 866 remaining studies, 319 had a recorded echo-based RAP estimate. These studies represented the “golden” data for which there was a matched set of a sniff video, a cardiologist's RAP estimate based on this sniff, and a ground-truth RAP measurement from right heart catheterization. These scans were always kept in the test set for all model training to avoid contamination. The 547 studies with a sniff scan and an RHC measurement but no RAP estimate were used as training and validation data for model fine-tuning, as described below.

The set of 15830 studies with a cardiologist-generated RAP estimate was divided into 12664 training studies, 1583 validation studies, and 1583 test studies. All 319 “golden” studies mentioned above with RHC measurements were included in the test set; otherwise, allocation was random. All models were constructed with a length-3 output layer and softmax activation to generate probabilities for 3 classes: 3 mmHg, 8 mmHg, and 15 mmHg. Training was performed on an NVIDIA RTX 6000 Ada graphics card.

Multiple modern video processing machine learning architectures were applied to the problem, including X3D, SlowFast, MoViNet, TimeSformer, STAM, and ViViT. It was observed that the model type that generated the highest validation performance within GPU memory constraints was SlowFast R50, so this architecture was selected for further tuning and evaluation. Model implementation was performed using the PyTorchVideo package.

When training the model, input pre-processing was applied in the following steps.
1. Standardize data length by selecting 64 frames evenly spaced throughout the length of the video. For a 10 second clip length (which is relatively long for this dataset) this will provide over 6 frames per second, which should guarantee catching a sniff. If the video is less than 64 frames long, pad at the end with empty frames.
2. Rescale each frame in the video such that the physical size of each pixel is 0.122 cm (the median for the whole dataset). If zoom in is needed, center crop. If zoom out is needed, pad the edges with the mean brightness of the image. The final size of all frames was kept at 224×224 pixels.
3. Apply random rotation and brightness/contrast adjustment transforms.
4. Convert the video to grayscale. Since the color Doppler scans were excluded, the underlying data is already grayscale, so going from 3 color channels to 1 cuts down on data size without losing information.
5. Normalize each frame in the video to have mean intensity 0 and standard deviation 1.

Regularization was performed via data augmentation as well as the architecture default parameters of batch normalization and 50% dropout in the final classification layer. In addition, label smooth was applied to the output targets. With this scheme, the targeted probability distribution is no longer 100% on the single correct answer; instead, some percentage is redistributed to the other categories. In noisy systems such as this one, label smoothing discourages the model from assigning unrealistically high certainty to any one answer. Typically, the smoothed probability is distributed evenly to all non-target classes. However, in this system some domain knowledge about which forms of noise were most likely was known; a scan labeled as 3 may properly have been an 8, but it was almost certainly not a 15. Thus, if label smoothing strength was 0.1, target probability distributions for 3, 8, and 15 were assigned as (0.9, 0.1, 0.0), (0.05, 0.9, 0.05), and (0.0, 0.1, 0.9), respectively.

Hyperparameter optimization was performed over number of frames, frame sampling stride, slow lane downsampling ratio, optimizer type, whether or not to apply grayscale, data augmentation strength, and label smoothing strength. Final parameters selected were: 64 frames, even sampling throughout the video, 8× slow lane downsampling, RAdam optimization, grayscale application, 50∘/10% data augmentation strength, and 0.1 label smoothing strength.

Models were trained for 100 epochs with categorical cross-entropy loss. Loss for each target class was weighted inversely to the frequency of that class in the dataset; e.g., loss for a video with target class 15 was multiplied by roughly 10 compared to loss for a video with target class 3. This encouraged the model to evenly distribute probabilities across the 3 classes if it was unsure, rather than guessing the most common class; without this scaling, models tended to get stuck in a local minimum of assigning label 3 to everything. The epoch which produced the highest validation accuracy was selected as the best model state, and these validation performances were compared to select the best set of hyperparameters. Out-of-sample performance from the best model was not measured until after hyperparameter optimization was completed.

While training a model to replicate cardiologist estimates of RAP can only hope to match human performance; RHC data gives the potential to surpass human performance. However, the dataset of 438 train scans and 109 validation scans with associated RHC data was too small to train a new machine learning model from scratch. To address this transfer learning was used to take advantage of all of the information about processing sniff scans that had already been learned from training to match cardiologist evaluations. To do this end, the best classification model was modified by replacing its output layer with a 1-dimensional output with no activation in order to convert it into a regression model. This new model was then trained to match RHC measurements. The optimized hyperparameters from before were kept, but there were two more factors to tweak; layer freezing and loss function.

The first consideration was layer freezing. The goal of transfer learning is to get the model to utilize its prior information about common features in the system to generate answers in new ways. If the entire model is trained normally, instability in the new randomly initialized output layer may propagate backwards and erase feature knowledge in earlier layers. The extreme solution to this is to freeze all pre-existing layers, such that only the weights of the final output layer can be changed. However, in the described system which had been pre-trained to replicate the sniff test, this type of freeze would have prevented any discovery of new useful features not related to existing standards. After testing various compromises between these two extremes, it was observed that a strategy that produced the best validation error in this particular implementation was training the whole model, but assigning a three-times lower learning rate to all pre-existing layers compared to that of the output layer. This allowed the model to learn to make use of existing features without erasing them while maintaining the possibility of morphing these features to better fit the new data.

The second consideration was the loss function. The standard loss function for regression tasks is mean squared error, or L2. However, this loss function was not well suited for this problem. To see why, consider a data point with a target of 11 mmHg and two different predictions; 4 mmHg or 21 mmHg. L2 loss would penalize the second prediction twice as strongly as the first, as the squared error would be 100 mmHg² compared to 49 mmHg². However, from a clinical standpoint, the first error is quite significant as someone with a problematic RAP has been given a healthy prediction. The second error, in contrast, is less significant, as both the prediction and target are unhealthy pressures that would likely lead to similar courses of treatment. To address this, a LeakyReLU transform was applied to soft-cap outputs and targets to the (2, 15) mmHg range; beyond that, being extra-low or extra-high does not have much clinical significance. Thereafter, a log transform was applied to the outputs and targets before getting L2 loss, reflecting the fact that each incremental increase in pressure is more clinically significant at the lower end of the spectrum.

With these adjustments in place, RHC-based training of the model was performed for 50 epochs, with early stopping based on best validation accuracy when converting predictions and targets back into 3/8/15 bins. To reduce noise in validation performance, the 109 validation scans were augmented five-times with random rotations and brightness/contrast scaling.

The first phase of model training and evaluation relied only on TTE videos of sniff tests and cardiologists' interpretations of those sniff tests. The model with the best validation performance, as measured by average classification accuracy across the three target classes, was run on the 1583 test scans to gauge out-of-sample performance. Table 3 shows the out-of-sample performance of the echo-based RAP classification model, evaluated on the full test set of 1583 scans.

Light gray boxes represent correct predictions, and the dark gray box represents a clinically problematic false negative prediction. The overall accuracy of 77.3% represents a weighted average of accuracy in each target class, with weights based on observed frequency of each class across the dataset. The false negative rate represents the percentage of patients classified as 15 mmHg by cardiologists (7.5% of the patient population) which the model classified as 3 mmHg.

Looking at the off-target predictions, it is worth noting that mis-classifying a targeted 3 as an 8 was significantly more likely than mis-classifying a targeted 3 as a 15, and similarly the 15→8 mis-call rate was higher than the 15→3 mis-call rate. This indicates that the model was able to learn that category 3 is “closer” to 8 than it is to 15, despite the fact that the model architecture treated all categories equally and independently with no inherent encoding of which ones were closer to each other. A similar pattern persisted even if label smoothing, the only bit of category-asymmetric information in the training procedure, was turned off.

For performance evaluation, it is also worth considering that not all wrong answers carry the same clinical significance. If a model like this were deployed as a screening tool in a clinical setting, the most problematic error would be a false negative, i.e., analyzing a scan from a patient who truly had an RAP in the 15 mmHg range and placing that patient in the 3 mmHg category. This could result in a patient who needed immediate treatment for volume overload instead being evaluated as healthy and not given follow-up testing or treatment. From this perspective, these results show a false negative rate 16.5%. The ideal screening model would have a higher sensitivity for 15 mmHg predictions and a correspondingly high specificity for 3 mmHg predictions.

As mentioned above, roughly 25% of this test set included scans in which the IVC was either obscured or not present at all. For obscured IVCs the model may have been able to extract some information, but for scans with no IVC at all the model was essentially forced to guess at the answer. It was hypothesized that these guesses could be a significant contributor to model errors, particularly the false negative rate. This hypothesis was tested by evaluating model performance on both a manually trimmed test set, which eliminated these 25% of scans without a clear IVC, and the strictly filtered “high-quality” set (described above) which only kept the 35% of scans with the highest scores from the IVC classification model. RAP prediction performance from both of these datasets is shown below in Table 4 (manually trimmed test set) and Table 5 (high-quality IVC test set).

As depicted, out-of-sample performance of the echo-based RAP classification model, increased only slightly from 77.3% originally to 77.5% on the trimmed set and 80.3% on the high-quality set. However, the clinically problematic false negative rate went down substantially, from 16.5% originally to 9.3% on the trimmed set and 5.6% on the high-quality IVC set.

Both dataset reduction procedures were blinded to any RAP information (either cardiologist estimates or model predictions), so this should accurately represent the performance expected from higher-quality data inputs. Obtaining this higher quality data would be possible in a clinical setting, as the operator could trigger machine learning-based RAP classification only once the IVC classification model indicated that a good view of the IVC had been obtained.

Further examination of the confusion matrices in Tables 4-5 shows that the extreme false positive rate (true 3 mmHg classified as 15 mmHg by the model) also fell as data quality increased. As a result, in the high-quality test set only 6% of incorrect predictions were in the 3→15 or 15→3 categories; the remaining 94% were off by 1. Some portion of these “errors” were likely due to human variability in generating the target estimates rather than underlying deficiencies in the model. For example, medical guidelines give a range of possible locations for measuring the diameter of the IVC, but the IVC generally does not have a constant diameter over this range. If the diameter changed from 20 mm at one end to 22 mm at the other, the “correct” RAP classification may not be clear to a physician, and different doctors may reasonably come to different conclusions. Indeed, prior studies which had the same IVC scans independently analyzed by multiple trained physicians to assess diameter and CI have found significant inter-operator variability, and those that specifically looked at inter-operator agreement rates for RAP assessment found agreement rates of 70-75%. This indicates that the model performance may be nearing the limit set by underlying uncertainty in the target data.

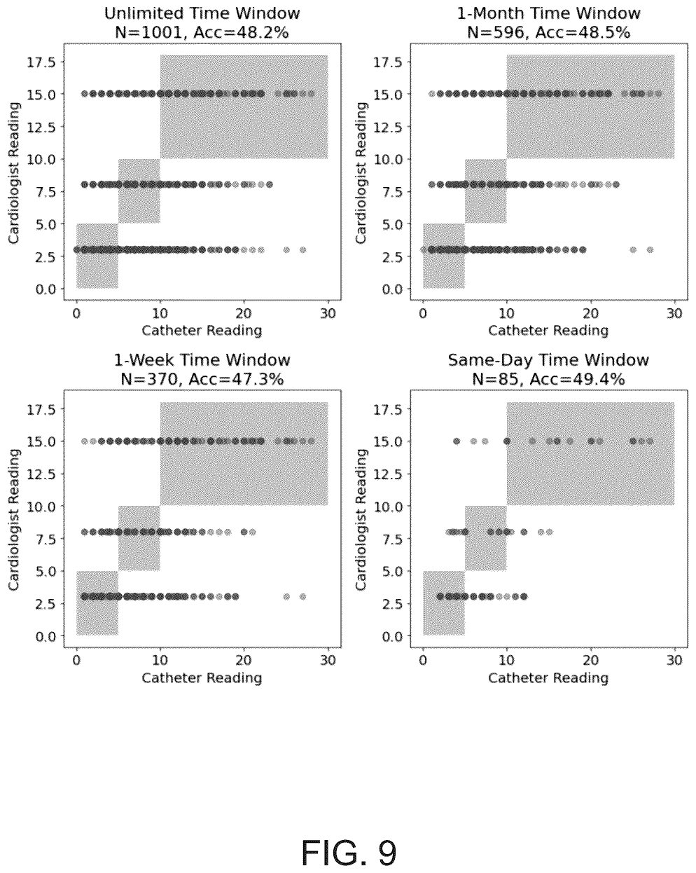

As indicated above, right heart catheter measurements provided a means of assessing model performance in a way that was independent of human interpreter variability. One limitation of the RHC data, as discussed above, is that the RHC and TTE measurements were generally not taken on the same day, and the patient's true RAP may have changed in the intervening time. The effect of this variance was observed by analyzing the accuracy of cardiologists' sniff-based assessments of RAP compared to RHC measurements with different thresholds of allowed time separation between the two measurements. This is depicted by FIG. 9, which includes plots showing the accuracy of cardiologist RAP estimates compared to true RAP values from right heart catheterization, with various time windows allowed between cardiologist and RHC measurements: unlimited time, 1 month, 1 week, or same day. The circles represent individual measurement pairs, and the shaded boxes are the “correct” prediction ranges.

As expected, widening the allowable time window significantly increased the total number of data points, from 85, which occurred on the same day to 596 which occurred within 30 days of each other. Somewhat unexpectedly, however, it was observed by the inventors that accuracy did not drop very sharply as the time window was widened, going from 49.4% for same-day measurements to 48.2% for all measurements with any time separation. As such, reducing the time window significantly reduced the amount of data available, but surprisingly only moderately increased prediction accuracy. This indicates that variation in true RAP between RHC and TTE measurements was not a significant error contributor in the dataset. Considering the tradeoff between dataset size and accuracy, a 30-day time window was chosen in this particular embodiment. While the width of this window created some risk of inaccuracy in the gold standard reference, errors should be reflected equally in both cardiologist and model predictions since they were both made based on the same TTE data.

The dataset was evaluated in comparison to a prior study which was specifically designed to evaluate the accuracy of the sniff test. In the prior study, 153 patients had their RAP measured by both RHC and a sniff test in quick succession. The sniff test was used to generate an RAP estimate based on multiple different standards which have previously been proposed. A summary of the results of this study, compared to RHC and TTE data with a 30-day time window applied, is shown below in Table 6 (prior study dataset) and Table 7 (30-day window dataset).

It would be expected that the prior study dataset would yield better results, as the sniff test and RHC were always conducted in quick succession in a single standardized manner. Surprisingly, however, the dataset utilized by the inventors actually showed higher overall accuracy and an effectively equivalent false negative rate, despite the time gap between sniff test and RHC measurements. This could plausibly be due to differences in patient populations or greater experience of the dataset's cardiologists in applying the specific sniff test standards as compared to physicians in the other study who were evaluating multiple different sniff test standards. Regardless of the reasons, the favorable accuracy levels of the dataset compared to prior literature provide further evidence that this dataset can be effectively used for evaluating machine learning model performance.

Before using the RHC data for further model training, the performance of the best echo-only model, which was trained to match cardiologist estimates, was evaluated on the “golden” test set which had echo video data, a sniff-based RAP estimate from a clinical cardiologist, and an associated RHC measurement within 30 days. This performance is summarized in Table 8.

It was observed that even though the model only agreed with cardiologist predictions roughly 77% of the time in the original test set (see Table 3), the performance of the model with respect to RHC results was effectively equivalent to that of the cardiologists, both in terms of overall accuracy and the false negative rate. Applying a categorical chi-squared test with 8 degrees of freedom to compare the distribution of predictions between the cardiologists and echo-only machine learning model (using scipy.stats.chi2_contingency) yielded p=0.98, indicating that the two distributions are statistically indistinguishable. These results provide strong evidence that the machine learning model effectively replicated the performance of cardiologists in analyzing sniff tests.

Performance was further enhanced by taking the best echo-only model and fine-tuning it using the smaller set of training data with RHC measurements. The results from this fine-tuned model are shown in Table 9.

It was observed that while fine-tuning had little effect on overall accuracy, there was a drastic change in the false negative rate, which dropped by over 70% compared to either cardiologist estimates or the echo-only machine learning model. This led to an overall improvement in the accuracy of classifying patients in the 15 mmHg category, which went from 45.4% with cardiologist estimates to 59.8% with the fine-tuned machine learning model. Such an improvement would represent a substantial decrease in the number of patients with elevated RAP being falsely given a clean bill of health.

Another way to evaluate the results is to consider how often each measurement method yielded results of 3, 8, or 15 mmHg, as depicted in Table 10.

TABLE 10

| | 3 mmHg | 8 mmHg | 15 mmHg |
|---|---|---|---|
| Measurement Type | Frequency | Frequency | Frequency |
| RHC | 37.0% | 32.6% | 30.4% |
| Cardiologist | 52.0% | 24.8% | 23.2% |
| Echo-Only machine learning | 48.6% | 28.2% | 23.2% |
| Fine-Tuned machine learning | 20.7% | 48.0% | 31.3% |
| RHC (prior study) | 11.1% | 44.4% | 44.4% |
| Cardiologist (prior study) | 37.9% | 34.0% | 28.1% |

The true distribution for the patient population can be read from the RHC results, which showed a roughly equal proportion across the three classes. As shown, cardiologists tended to underestimate RAP from the sniff test, with over half of patients assessed as 3 mmHg and fewer than a quarter assessed as 15 mmHg. This pattern of underestimation was replicated in the prior study's results, indicating that it is not just an artifact of the dataset used in this particular embodiment. The Echo-Only machine learning model that was trained to match cardiologist predictions, unsurprisingly, exhibited an equivalent tendency towards underestimation. The fine-tuned machine learning model, in contrast, over-corrected a bit, exhibiting underprediction of 3 mmHg and overprediction of 8 mmHg. If such a machine learning model were used as an initial patient screening tool upon admission, this tendency towards overestimation may be preferable, as an overly high RAP estimate from the model would be resolved after the patient was referred for further testing while an underestimate may go undetected if the patient is deemed healthy and not referred for further testing.

### Broader Heart Health Integrative Modeling

Although the disclosure has focused on automated evaluation of sniff test scans via machine learning to estimate RAP, it is anticipated by the inventors that some of the machine techniques described herein could potentially be generalized to provide a more comprehensive evaluation of heart health. When cardiologists evaluate a patient they generally perform a more comprehensive set of scans covering all parts of the heart and surrounding vessels from many angles, known as a TTE study. A broader model architecture could incorporate all of these scans to generate broader and more accurate information about heart health.

To this end, a broader model architecture for evaluating heart health can be developed as follows. One or more encoder models can be trained to transform the different scans associated with TTE scan (which may consist of any view of the heart) into fixed-dimension vectors. For example, a first scan associated with first heart information can be transformed, using a first encoder model, into a first lower-dimensional tensor, and a second scan associated with second heart information can be transformed into a second lower-dimensional tensor using a second encoder model. Contrastive learning methods can be used to perform unsupervised training on a large dataset of TTE scans from one or more medical centers such that similar scans get transformed into similar vectors. This is similar to word embeddings used by existing language models. Any suitable encoder model architecture could be used, but the SlowFast architecture could be employed in some embodiments as this architecture has been found to work well for TTE scans.

Next, a transformer model can be trained to provide an overall assessment of heart health from an encoded TTE scan. In some implementations, all of the encoded “words” (i.e., individual, encoded tensors from multiple different measurements of the heart of the same subject) from each scan in a TTE study could be grouped into an unordered “sentence”. A transformer-type model can be created/trained to convert this “sentence” into a final heart state vector of fixed-dimension (e.g., a new single tensor) encoding the overall health state of the patient's heart. This transformer model can also undergo unsupervised training using contrastive learning.

In some implementations, supervised training could be used to create separate models that analyze a heart state vector to generate many different measurements, such as RAP, which currently require manual evaluation by cardiologists.

In some implementations, non-ultrasound data such as right heart catheter measurements can be used to improve interpretation of TTEs beyond the currently capabilities of cardiologists. In some cases, non-ultrasound data such as right heart catheter measurements could be used to improve interpretation of TTEs beyond the currently capabilities of cardiologists.

Although the aforementioned type of training may require large quantities of data to generate results, it is possible given the large number (e.g., millions) of TTE scans and other medical imaging studies that may be present in the databases of large medical centers. An advantage of this approach is that it can generate predictions using information from the whole heart and can be based off of a variable number of scans. This could represent a significant improvement over standard medical machine learning applications that try to generate a fixed measurement based on a fixed data input. It should be appreciated that the aforementioned generalized method need not be limited to ultrasound imaging data alone; given a suitably sized dataset it could easily incorporate cardiac information from ECGs or other sources. For example, models that encode different types of cardiological data (e.g., an ultrasound scan, an ECG reading, a CT scan, etc.) could generate tensors that are combined using a transformer model as described above to generate the heart state vector.

In this document, the terms “machine readable medium,” “computer readable medium,” and similar terms are used to generally refer to non-transitory mediums, volatile or non-volatile, that store data and/or instructions that cause a machine to operate in a specific fashion. Common forms of machine readable media include, for example, a hard disk, solid state drive, magnetic tape, or any other magnetic data storage medium, an optical disc or any other optical data storage medium, any physical medium with patterns of holes, a RAM, a PROM, EPROM, a FLASH-EPROM, NVRAM, any other memory chip or cartridge, and networked versions of the same.

These and other various forms of computer readable media may be involved in carrying one or more sequences of one or more instructions to a processing device for execution. Such instructions embodied on the medium, are generally referred to as “instructions” or “code.” Instructions may be grouped in the form of computer programs or other groupings. When executed, such instructions may enable a processing device to perform features or functions of the present application as discussed herein.

In this document, a “processing device” may be implemented as a single processor that performs processing operations or a combination of specialized and/or general-purpose processors that perform processing operations. A processing device may include a CPU, GPU, APU, DSP, FPGA, ASIC, SOC, and/or other processing circuitry.

The terms “substantially” and “about” used throughout this disclosure, including the claims, are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

To the extent applicable, the terms “first,” “second,” “third,” etc. herein are merely employed to show the respective objects described by these terms as separate entities and are not meant to connote a sense of chronological order, unless stated explicitly otherwise herein.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof, the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

It should be appreciated that all combinations of the foregoing concepts (provided such concepts are not mutually inconsistent) are contemplated as being part of the inventive subject matter disclosed herein. In particular, all combinations of claimed subject matter appearing in this disclosure are contemplated as being part of the inventive subject matter disclosed herein.

What is claimed is:

1. A non-transitory computer-readable medium having executable instructions stored thereon that, when executed by a processor, cause a system to perform operations comprising:
obtaining first video scan data comprising multiple first video frames of an inferior vena cava (IVC) of a subject, the multiple first video frames including a first video frame of the IVC while the subject is at rest, and a second video frame of the IVC while the subject is inhaling;
determining, using a first trained model, based at least on the multiple first video frames, that the first video scan data corresponds to a sniff test of the IVC of the subject; and
predicting, using a second trained model, based at least on the multiple first video frames, a right atrial pressure (RAP) of the subject.

2. The non-transitory computer-readable medium of claim 1, wherein predicting the RAP of the subject is performed in response to determining that the first video scan data corresponds to a sniff test of the IVC of the subject.

3. The non-transitory computer-readable medium of claim 1, wherein:
the operations further comprise:
prior to obtaining the first video scan data, obtaining second video scan data comprising multiple second video frames of the IVC; and
determining, using a third trained model, based at least on the multiple second video frames, that an acceptable view of the IVC is being captured; and
obtaining the first video scan data comprises obtaining the first video scan data in response to determining that the acceptable view of the IVC is being captured.

4. The non-transitory computer-readable medium of claim 3, wherein: determining, using the third trained model, based at least on the multiple second video frames, that an acceptable view of the IVC is being captured, comprises:
generating, using the third trained model, based at least on the multiple second video frames, a prediction comprising a confidence score that indicates a likelihood that the second video scan data is associated with an IVC class; and
in response to determining that the confidence score meets a threshold, making a determination that an acceptable view of the IVC is being captured.

5. The non-transitory computer-readable medium of claim 3, wherein obtaining the first video scan data and the second video scan data comprises: capturing, using an ultrasound imaging device, a transthoracic echocardiogram of the subject.

6. The non-transitory computer-readable medium of claim 3, wherein the operations further comprise:
obtaining multiple video scans, each of the multiple video scans corresponding to a medical imaging study;
assigning multiple labels to the multiple video scans, each of the labels indicating whether or not a respective one of the video scans corresponds to a view of an IVC; and
training, based on the multiple video scans and the multiple labels, a classification model as the third trained model.

7. The non-transitory computer-readable medium of claim 1, wherein: determining, using the first trained model, based at least on the multiple first video frames, that the first video scan data corresponds to the sniff test comprises:
generating, using the first trained model, based at least on the multiple first video frames, a prediction comprising a confidence score that indicates a likelihood that the first video scan data is associated with a sniff test class; and
in response to determining that the confidence score meets a threshold, making a determination that the first video scan data corresponds to the sniff test of the IVC of the subject.

8. The non-transitory computer-readable medium of claim 1, wherein the operations further comprise:
obtaining multiple video scans, each of the multiple video scans corresponding to a medical imaging study;
assigning multiple labels to the multiple video scans, each of the labels indicating whether or not a respective one of the video scans corresponds to a view of an IVC during a sniff test; and
training, based on the multiple video scans and the multiple labels, a classification model as the first trained model.

9. The non-transitory computer-readable medium of claim 1, wherein predicting, using the second trained model, based at least on the multiple first video frames, the RAP of the subject comprises:
inputting the multiple first video frames into the second trained model; and
generating, using the second trained model, a prediction output including the RAP.

10. The non-transitory computer-readable medium of claim 9, wherein the RAP of the prediction output is between 0 mmHg and 30 mmHg.

11. The non-transitory computer-readable medium of claim 9, wherein the RAP of the prediction output is 3 mmHg, 8 mmHg, or 15 mmHg.

12. The non-transitory computer-readable medium of claim 1, wherein the operations further comprise:
obtaining multiple video scans, each of the multiple video scans corresponding to a sniff test of a subject;
obtaining multiple RAP measurements, each of the RAP measurements corresponding to a respective one of the video scans; and
constructing, based on the multiple video scans and the multiple RAP measurements, the second trained model.

13. The non-transitory computer-readable medium of claim 12, wherein:
the multiple video scans were captured by a plurality of different models of ultrasound imaging machines; and
the multiple RAP measurements comprise a plurality of RAP estimates made by a plurality of different cardiologists.

14. The non-transitory computer-readable medium of claim 12, wherein each RAP measurement of the RAP measurements is an RAP estimate made by a physician based on the respective one of the video scans corresponding to the RAP measurement.

15. The non-transitory computer-readable medium of claim 12, wherein each RAP measurement of the RAP measurements is a right heart catheterization (RHC) measurement made via RHC of a subject.

16. The non-transitory computer-readable medium of claim 15, wherein each of the RHC measurements corresponds to a respective one of the video scans made of a same subject within one month or less.

17. The non-transitory computer-readable medium of claim 12, wherein:
the multiple video scans comprise a first plurality of video scans and a second plurality of video scans;
the multiple RAP measurements comprise a first plurality of RAP measurements and a second plurality of RAP measurements;
each of the first plurality of RAP measurements is an RAP estimate made by a physician based on a respective one of the first plurality of video scans; and
each of the second plurality of RAP measurements is a RHC measurement made via RHC of a subject, and corresponds to a respective one of the second plurality of video scans made of a same subject.

18. The non-transitory computer-readable medium of claim 17, wherein constructing, based on the multiple video scans and the multiple RAP measurements, the second trained model, comprises:
pre-training, based on the first plurality of video scans and the first plurality of RAP measurements, an input model to estimate RAP based on an input video scan; and
applying, based on the second plurality of video scans and the second plurality of RAP measurements, transfer learning to the input model to construct the second trained model.

19. The non-transitory computer-readable medium of claim 18, wherein applying transfer learning to the input model to construct the second trained model comprises:
replacing an output layer of the input model with a new output layer to produce a new model; and
training, using the second plurality of video scans and the second plurality of RAP measurements, the new model.

20. The non-transitory computer-readable medium of claim 19, wherein training the new model comprises assigning a lower learning rate to pre-existing layers of the input model present in the new model compared to the new output layer.

21. A system, comprising:
a processor; and
a non-transitory computer-readable medium having executable instructions stored thereon that, when executed by the processor, cause the system to perform operations comprising:
obtaining first video scan data comprising multiple first video frames of an inferior vena cava (IVC) of a subject, the multiple first video frames including a first video frame of the IVC while the subject is at rest, and a second video frame of the IVC while the subject is inhaling;
determining, using a first trained model, based at least on the multiple first video frames, that the first video scan data corresponds to a sniff test of the IVC of the subject; and
predicting, using a second trained model, based at least on the multiple first video frames, a right atrial pressure (RAP) of the subject.

22. The system of claim 21, further comprising: an ultrasonic imaging device configured to capture the first video scan data.

23. The system of claim 22, wherein the ultrasonic imaging device is a portable ultrasonic imaging device.

24. The system of claim 23, wherein:
the processor and non-transitory computer-readable medium are components of a mobile device; and
the mobile device is communicatively coupled to the portable ultrasonic imaging device.

25. The system of claim 21, wherein the system is a mobile device.

26. The system of claim 21, wherein the operations further comprise: displaying, on a graphical user interface, the RAP that is predicted.

27. A method, comprising:
obtaining, at a computing device, first video scan data comprising multiple first video frames of an inferior vena cava (IVC) of a subject, the multiple first video frames including a first video frame of the IVC while the subject is at rest, and a second video frame of the IVC while the subject is inhaling;
determining, at the computing device, using a first trained model, based at least on the multiple first video frames, that the first video scan data corresponds to a sniff test of the IVC of the subject; and
predicting, at the computing device, using a second trained model, based at least on the multiple first video frames, a right atrial pressure (RAP) of the subject.
